(19) United States
(12) Patent Application Publication
(10) Pub. No.: US 2016/0169884 A1
(43) Pub. Date: Jun. 16, 2016

(54) DEVICE AND METHOD FOR BIOLOGICAL ANALYSES

(71) Applicant: BIOMÉRIEUX, Marcy-I'Etoile (FR)

(72) Inventor: Nathalie RENARD, Brignais (FR)

(21) Appl. No.: 14/909,697

(22) PCT Filed: Jul. 31, 2014
(86) PCT No.: PCT/FR2014/052004

§ 371 Date: Feb. 2, 2016

(30) Foreign Application Priority Data
Aug. 2, 2013 (FR) 1357754

(51) Int. Cl. G01N 33/543

(52) U.S. Cl. CPC G01N 33/54386

(57) ABSTRACT

A device, method and use aimed at the biological control of the binding partners of an analyte within the framework of a biological analysis.

### TECHNICAL FIELD

The present invention concerns the field of detection and/or quantification of analyte(s) in liquid samples during a biological analysis. The aim of the present invention is, notably, a device and a method for performing a sandwich-type immunoassay, in order to detect and/or quantify at least one analyte in a sample. In particular, the invention enables biological control (functional/operational character control) of the analyte binding partners, used within the framework of said sandwich-type immunoassay, in order to prevent false negative results from being obtained when all or some of the binding partners used are defective.

### STATE OF THE ART

Sandwich-type immunoassays are widely employed in bioanalysis, in order to detect and/or quantify a given analyte. These sandwich-type immunoassays (or more simply “sandwich immunoassays”) use a first binding partner, known as the “capture binding partner”, generally immobilized on a solid phase to bind, specifically, the analyte sought, and a second binding partner, known as the “detection binding partner”, labelled and also designed to bind specifically to the analyte sought, thereby revealing the bond between the capture binding partner and the analyte, and therefore the presence of the analyte. In other words, the analyte sought finds itself “sandwiched” between said first and second binding partners, the first (“capture”) binding partner generally being present in excess in relation to the analyte sought. The capture binding partner may, for example, be immobilized on a solid support (by covalent bonding, adsorption or any other appropriate method), and the quantity must generally be such that the number of binding sites present on the capture binding partner is greater than the number of antigen molecules present in the standard or unknown solutions. The labelled detection binding partner may, for example, be added, either simultaneously, or after initial incubation and washing, and attaches to the antigen, previously immobilized on the capture binding partner.

A simple washing operation is used to separate the capture binding partner-analyte-(labelled) detection binding partner complexes from the free labelled detection binding partners, present in excess.

For the labelled detection binding partner to be able to bind to the analyte already engaged in a reaction with the capture binding partner, it is necessary for both binding partners to recognise different areas of the analyte.

The labelling of the detection binding partner may be, for example, by means of a radioactive isotope, or enzymatic. In any event, in the various labelling scenarios, the signal corresponding to the capture binding partner-analyte-labelled detection binding partner complex is a signal representative of the quantity of analyte(s) present in the tested sample.

Traditionally, in sandwich-type immunoassays, the capture and detection binding partners are antibodies recognising epitopes different from the analyte sought, which, in this embodiment, consists of an antigen. The antibodies used as the capture binding partner (“first binding partner”) and the detection binding partner (“second binding partner”) may be, for example, multivalent polyclonal antibodies or various specific monoclonal antibodies.

The use of two binding partners (for example antibodies, such as monoclonal antibodies) possessing recognition specificity for two different sites of the analyte (for example two distinct epitopes of an antigen) lends this method very good sensitivity. Thus, to obtain a “false positive” corresponding to a detected molecule confused with the analyte of interest, this molecule must possess the same two epitopes as the analyte sought. By correctly selecting said epitopes, interference with other highly homologous molecules can therefore be extremely significantly reduced.

Sandwich-type immunoassays, by virtue of their high sensitivity, may form the basis for various types of test. As an illustration, mention can be made of, within the framework of EIA (“Enzyme ImmunoAssays”), the sandwich ELISA test (“Enzyme-Linked immunosorbent assay”. Broadly speaking, this test proceeds as follows:
a. a surface (for example a plate, such as a “96-well” plate) is covered with a quantity of so-called “capture” antibodies;
b. the sample capable of containing the antigen sought is added, such that any antigen present binds to the capture antigen;
c. the plate is washed so as to eliminate all non-fixed molecules other than the analyte;
d. the so-called “detection” antibody, directly or otherwise enzyme-conjugated, is added and binds to the antigen, thereby forming a capture antibody-antigen-detection antibody complex;
e. the plate is rinsed so as to separate this complex from the free labelled detection antibodies, present in excess;
f. where applicable, the chromogenic or fluorogenic enzyme substrate (this is then referred to as an “ELFA” assay) is added; the latter is converted by the enzyme into a detectable form (coloured or fluorescent).

The result of this “ELISA sandwich” test can be analysed by the naked eye or in a spectrophotometer specially designed to directly accept the plates (for example “96-well” plates).

Sandwich-type immunoassays can be employed, advantageously, using “Magnotech” technology, from Philips. A test of this type, namely using “Magnotech” technology, is notably described in the publication by Bruls et al. [1]. This technology employs a reaction cartridge, preferably made of plastic, comprising a liquid sample application area and one or more reaction chambers comprising the various reagents required for the test, this reaction area being connected to the sample application area by a channel enabling capillary flow of the liquid sample from the application area toward said reaction area. Substantially, the reaction area notably comprises magnetic nanoparticles (of around one hundred nanometres in diameter, for example superparamagnetic particles), dried and coated with an antibody of interest capable of binding specifically to a first epitope of the analyte sought. Furthermore, said reaction area includes, in a region which can be characterized as a “detection region”, antibodies capable of specifically recognising a second epitope of the antigen sought. Optimally, this reaction cartridge can be placed within a portable device comprising two electromagnets positioned above and below the reaction cartridge. The immunoassay result is read after magnetic actuation, with the upper electromagnet serving as a washing magnet, while the lower electromagnet serves as a binding magnet. Such technology is notably described in patent application WO2013/054230.

As described in Bruls et al. [1] or in patent application WO2013/054230, the presence of said complexes in the detection region—and therefore of the antigen sought—is detected optically, and more specifically, is based on the principle of frustrated total internal reflection (f-TIR). The detection principle is as follows: in the absence of magnetic nanoparticles in the detection region, the incident light beam is reflected with maximum intensity. If there are nanoparticles present in the detection region, part of the incident light is reflected and diffused by these nanoparticles, resulting in decreased intensity of the reflected light beam. This signal reduction is proportional to the number of nanoparticles bound to the detection region of the reaction area, and consequently, to the concentration of analyte sought in the tested sample.

This technique also makes it possible to quantify the analyte sought by plotting a calibration curve based on the optical signal variation as a function of the concentration of magnetic nanoparticles, and therefore indirectly of the analyte.

Several applications of the “Magnotech” system have been described in the literature, for example, a “point-of-care” (POC) test with a total duration of five minutes to detect the cardiac protein troponin I (cTnI) in a sample of a patient's whole blood (see notably Bruls et al. [1] and Dittmer et al. [2]). Cardiac troponin I is a reference standard in the framework of severe myocardial infarction diagnostics, as described in Morrow et al. [3].

Jarrige et al. [4] describe an application of the “Magnotech” technology platform for assaying the “Intraoperative parathyroid hormone” (ioPTH) via plotting a dose-response curve of the parathyroid hormone (PTH).

However, the issue of biological control (functional/operational character control) of the binding partner immobilized on the magnetic nanoparticles and/or of the binding partner immobilized on the reaction area detection region is not covered within the framework of the tests described above, employing the “Magnotech” platform. The absence of variation of the measured signal is therefore only indicative of the absence of the analyte sought, whereas it could be due to a defect in one or more of the above-mentioned two binding partners. In the latter case, since the apparent test result would be negative, the user would therefore, wrongly, conclude that the analyte sought is not present in the tested sample. A result of this type is known as a “false negative”, and can prove extremely harmful to the health of the patient tested, in particular if the analyte sought is an indicator of a serious pathology, requiring an emergency treatment, as is the case with cardiac troponin I.

More commonly, sandwich-type immunoassays are employed within the framework of unit tests, for example in rapid unit tests, such as rapid screening tests (RST). The latter generally come in the form of a “cassette”, like pregnancy tests, and are relatively easy to use. These tests are generally based on the principle of immuno-chromatography or membrane filtration: the sample (for example whole blood, serum, urine) deposited onto the support will, for example, migrate by capillarity, carrying with it some reagents already present, and will then encounter binding partners deposited onto the membrane when the sample is filtered by this membrane. These rapid unit tests are also referred to as “lateral flow assays” or “lateral flow tests”, and have been abundantly described in the literature (see for example Yager et al. [5] or Wild [6]). They are commonly used for the purposes of clinical, pharmaceutical, food and chemical analyses. Thus, lateral flow test devices can be employed to determine the presence of numerous types of analytes, such as antibodies, antigens, hormones, proteins, and chemical molecules, present in liquid samples.

Roughly, within the framework of a lateral flow test, the analyte to be detected, if it is present in the liquid sample deposited in the application area, binds to a binding partner labelled in the labelling area, the complexes thus formed then migrate to the reaction area where they are immobilized in the capture area by binding to a capture binding partner. The user can detect the presence of the analyte sought by revealing a detectable signal, determined depending on the type of label associated with the detection binding partner. Generally, the presence of the analyte of interest in the sample is demonstrated in the form of a detectable line, usually referred to as a test line. The reaction area generally comprises a sample migration control region, which will indicate to the user that at least a proportion of the sample has passed through the matrix, upstream of the control region, and in particular in the capture area. This may be, for example, by revealing a control line of a predetermined colour. By way of example, mention may be made of patent applications WO 2004/003559, WO 2006/092103, WO 2007/081330, US 2004/0161859.

Patent application WO 2012/066235, in the name of the Applicant, discloses a device making it possible to perform a rapid unit test, said device incorporating a positive control. To this end, a control region is provided within this device, for example downstream of the results viewing area, or parallel thereto, said control region comprising at least one analogue of the analyte sought capable of binding to the labelled detection binding partner. In other words, the rapid unit test covered by application WO 2012/066235 makes it possible not only to detect the analyte sought, but also to test the functional/operational character of the detection binding partner. Thus, if this control proves negative, the result of this test is considered uninterpretable, and the test must be repeated. Although making it possible to obtain satisfactory results, the Applicant has discovered that the sensitivity of the device and the method covered by WO 2012/066235, and furthermore of sandwich-type immunoassays in general, could be significantly improved.

Indeed, the Applicant has discovered, against all expectation, that the functionality (functional/operational character) of the capture binding partner, generally immobilized/adsorbed on a solid surface, could notably be affected in full or part by external factors, such as light, temperature, etc. Within the framework of a device such as described within application WO 2012/066235 or within the framework of an immunoassay employing the “Magnotech” platform (see above), this generates “false negatives”, insofar as the analyte, although present in the tested sample, is not detected in the so-called “results viewing” area, but the control region makes it possible to confirm the functionality (functional/operational character) of the detection antibody, and consequently the apparent reliability of the test, when the capture partner is defective. The user/operator therefore, wrongly, concludes from this that the analyte sought is not present within the tested sample.

### STATEMENT OF THE INVENTION

That is why one object of the present invention concerns a device making it possible to detect and/or quantify at least one analyte in a liquid sample during a biological analysis employing at least two binding partners P1 and P2 of said analyte, the first binding partner P1 being immobilized within said device and the second binding partner P2 being immobilizable by formation of a sandwich-type complex with the analyte and the first binding partner P1, said device comprising at least two areas in fluid communication, namely:

a) a liquid sample application area, and
b) a reaction area for detecting and/or quantifying said at least one analyte, said device comprising at least a first analogue CTRL1 of said analyte, said first analogue CTRL1 being immobilizable by binding to the immobilized first binding partner P1, and at least a second analogue CTRL2 of the analyte, said second analogue CTRL2 being immobilized within said device so as to bind to the immobilizable second binding partner P2, and thereby immobilize the latter,
said reaction area b) comprising at least the three following regions:
b.1) a first region for detecting and/or quantifying the analyte, by revealing the formation of a sandwich-type complex between the first binding partner P1, the analyte and the second binding partner P2,
b.2) a second biological control region (functional/operational character control) for the first binding partner P1, in which the first binding partner P1 is immobilized, a bond between the first binding partner P1 and the first analyte analogue CTRL1 is revealed, indicating a positive control for the first binding partner P1, and
b.3) a third biological control region (functional/operational character control) for the second binding partner P2, in which the second analyte analogue CTRL2 is immobilized, a bond between the second binding partner P2 and the second analyte analogue CTRL2 is revealed, indicating a positive control for the second binding partner P2.

According to a particular embodiment, the device according to the invention comprises a liquid sample migration area c), enabling migration of the liquid sample from the liquid sample application area a) toward the reaction area b). The presence of this liquid sample migration area c) is particularly desirable if the device according to the invention is suitable for use in a rapid unit test. Preferably, the liquid sample migration area c) comprises a matrix.

Preferably, the first and second binding partners P1 and P2 consist in antibodies, and the first and second analyte analogues CRTL1 and CRTL2 comprise at least the epitopes recognized respectively by the first and second antibodies P1 and P2. Advantageously, the first and second analyte analogues CRTL1 and CRTL2 are peptides containing at least the epitopes recognized respectively by the first and second antibodies P1 and P2.

Advantageously, the second binding partner P2 and the first analyte analogue CTRL1 are initially deposited—advantageously deposited and dried—into the device, and are suitable for being re-suspended in the presence of the liquid sample.

According to a first embodiment of the invention, the reaction area b) of the device according to the invention comprises at least two distinct parts, for example two distinct reaction chambers, with no direct fluid communication between the two parts, the first part comprising the first region b.1) and the second part comprising the second and third regions b.2) and b.3). Advantageously, the first part comprises, besides the immobilized first binding partner P1, the immobilizable second binding partner P2, and the second part comprises, besides the first binding partner P1 and the second analyte analogue CRTL2, which are both immobilized, the first analyte analogue CTRL1 and the second binding partner P2, which are both immobilizable.

According to a second embodiment of the invention, the reaction area b) of the device according to the invention comprises at least two distinct parts, for example two distinct reaction chambers, with no direct fluid communication between the two parts, the first part comprising the first and third regions b.1) and b.3) and the second part comprising the second region and b.2). Advantageously, the first part comprises, besides the first binding partner P1 and the second analyte analogue CRTL2, which are both immobilized, the immobilizable second binding partner P2, and the second part comprises, besides the immobilized first binding partner P1 the immobilizable first analyte analogue CTRL1, preferably the immobilizable second binding partner P2 being present in excess in said first part.

According to a third embodiment of the invention, the reaction area b) of the device according to the invention comprises at least three distinct parts, for example three reaction chambers, with no fluid communication between the three distinct parts, the first part comprising region b.1), the second part comprising region b.2) and the third part comprising region b.3). Advantageously, the first part comprises, besides the immobilized first binding partner P1, the immobilizable second binding partner P2, the second part comprises, besides the immobilized second analyte analogue CTRL2, the immobilizable second binding partner P2, and the third part comprises, besides the immobilized first binding partner P1, the immobilizable first analyte analogue CTRL1.

If the reaction area b) comprises several parts, as is the case with regard to the above-mentioned first, second and third embodiments, these parts are positioned in parallel or in series on the liquid sample pathway, preferably in parallel.

According to a fourth embodiment of the invention, the reaction area b) of the device according to the invention comprises at least one part, for example a reaction chamber, said part comprising:
the three regions b.1), b.2) and b.3),
the second binding partner P2 and the first analyte analogue CTRL1, which are both immobilizable,
   the first and second binding partners P1 and P2 being present in excess, and wherein:
the first analyte analogue CTRL1 is associated, directly or indirectly, with a label M1 making it possible to reveal the bond of said first analyte analogue CTRL1 with the first binding partner P1 in regions b.1) and b.2), and/or
the second binding partner P2 being associated, directly or indirectly, with a label M2, different from label M1, making it possible to reveal the formation of the sandwich-type complex between the second binding partner P2, the analyte and the first binding partner P1 in regions b.1) and b.2),
   so as to distinguish the bond of said first analyte analogue CTRL1 with the first binding partner P1 from the formation of the sandwich-type complex between the second binding partner P2, the analyte and the first binding partner P1, in said regions b.1) and b.2).

Preferably, regardless of the embodiment of the invention in question, the liquid sample application area a) comprises a filter.

Another object of the invention concerns a method for detecting and/or quantifying at least one analyte in a liquid sample during a biological analysis employing at least two analyte binding partners P1 and P2, said method comprising the steps consisting in:

(i) placing the liquid sample in contact with a device as defined above,
(ii) interpreting the result obtained from said device if the biological control (functional/operational character control) of the binding partner P1 and the biological control (functional/operational character control) of the binding partner P2 are positive, respectively in regions b.2) and b.3),
(iii) otherwise, considering the result obtained as uninterpretable.

The invention also concerns the use of at least two analyte analogues CTRL1 and CTRL2 for the biological control (functional/operational character control), respectively, of at least two binding partners P1 and P2, said binding partners P1 and P2 making it possible to detect and/or quantify said analyte by revealing the formation of a sandwich-type complex between the second binding partner P2, the analyte and the first binding partner P1.

“Detecting . . . at least one analyte” is to be understood to mean detecting the presence of the analyte sought within the liquid sample of interest.

“Quantifying at least one analyte” is to be understood to mean determining the quantity (for example the concentration)/assaying the analyte sought within said liquid sample.

“Sample” is to be understood to mean a small part or small isolated quantity of an entity for analysis purposes. This can be a clinical sample, human or animal, from a specimen of biological fluid, or a food sample, from any type of food, or a sample from the food production or processing environment. As indicated above, this sample is liquid.

Sample of clinical origin is to be understood to mean a sample taken from a patient or individual (human or animal), and capable of containing an analyte as defined below. This sample can notably be a liquid biological sample, such as a sample of blood, serum, plasma, saliva, urine, cerebrospinal fluid, pleural fluid, or peritoneal fluid (non-exhaustive list).

As samples of food origin, mention can be made of, for example, a food sample of water, beverages such as milk or fruit juice, yoghurt, meat, eggs, vegetables, mayonnaise, cheese, fish, etc. This sample of food origin can also come from an animal feed, such as notably a sample from animal meals.

As samples of environmental origin, mention can be made of, by way of illustration, a sample for control of a surface area or water body, or also taken from effluents, sludges, soils, plants, etc.

The sample taken can be used as-is or, prior to the biological analysis, undergo preparation by enrichment, dilution, extraction, concentration or purification, in accordance with methods known to the person skilled in the art.

“Liquid” sample should be understood to mean samples taken directly in liquid form, but also semi-solid or solid samples, insofar as they can be transformed into a liquid sample by any appropriate method known to the person skilled in the art. Of course, when the sample is solid or semi-solid, it must be pretreated to be transformed into a liquid sample.

This sample is obtained through any type of sampling known to the person skilled in the art.

The term “analyte” must be understood, in the broad sense, as referring to a chemical, biological or biochemical substance which undergoes one or more analysis(es). As examples of analytes, mention can be made of an antigen, an antibody, a hormone, a protein or a chemical molecule (non-exhaustive list).

As is known to the person skilled in the art, the nature of the analyte sought will dictate the nature of the binding partners to use. For example, if the analyte sought is a protein or an antigen, it can be detected and/or quantified by binding partners such as receptors, antibodies, antibody fragments, antibody analogues and any other ligand capable of binding to a protein or to an antigen. “Antibody analogues” are to be understood to mean biological and/or chemical compounds which possess the same binding capacities as the antibodies or antibody fragments, or similar binding capacities. In particular, antibody analogues include small proteins which, like antibodies, are capable of binding to a biological target thus enabling it to be detected, captured or simply targeted within an organism or a biological sample. The application fields of these antibody analogues are practically as vast as those of the antibodies. By way of example, mention can be made of Nanofitins™, small proteins marketed by Affilogic.

“Analyte analogue” is to be understood to mean a chemical, biological or biochemical species which exhibits physico-chemical and biological properties (for example in terms of affinity for a given ligand) similar to those of the analyte. Preferably, the analyte analogue is a protein, such as an antibody, a mixture of proteins (for example a mixture of antibodies), a polypeptide, a mixture of polypeptides, a peptide, a mixture of peptides, an antibody fragment, a mixture of antibody fragments, an antibody analogue, a mixture of antibody analogues, or their associations.

Generally, if the binding partner is an antibody, an antibody fragment or an antibody analogue, the analyte is a protein, a polypeptide or a peptide, and the analyte analogue is also a protein, a polypeptide or a peptide.

The expression “parts . . . without direct fluid communication” must be understood, in the sense of the present application, as referring to parts (for example reaction chambers) suitable for receiving a fluid, in this case a liquid, said parts furthermore being suitable, once this liquid has been received, for the latter not to be able to communicate freely between said parts (for example between said reaction chambers). The absence of direct fluid communication may be ensured, for example, by partitioning the parts (for example reaction chambers) by means of leakproof partition(s) and, more generally, by any technical solution known to the person skilled in the art.

Thanks to the functionality biological control (functional/operational character) of the first and second binding partners, the device according to the invention possesses very good sensitivity, namely that the probability of obtaining a “false negative” result is extremely low, to the point of being practically or entirely non-existent. Sensitivity is to be understood to mean the ability to give a positive result if the analyte sought is present in the tested liquid sample.

The biological analysis employed by means of the device according to the invention covers any immunoassay employing at least two binding partners, such as a sandwich-type immunoassay (also known as sandwich-type immunological assay).

Of course, the prefix “immuno” in the term “immunoassay”, for example, should not be considered in the present application as strictly indicating that the binding partner is necessarily a partner of immonological origin, such as an antibody or antibody fragment. Indeed, as is well known to the person skilled in the art, this term is more widely used to refer to tests and methods wherein the binding partner is not a partner of immunological origin/nature, but consists, for example, in a receptor of the analyte which it is desired to detect and/or quantify. The condition being that the binding partner concerned is able to bind to the analyte sought, preferably specifically. Thus, it is known to speak of the ELISA assay for assays which use non-immunological binding partners in the strict sense, more widely called “ligand binding assay”, whereas the term “immuno” itself is included in the full version of the acronym ELISA. For the sake of clarity and uniformity, the term “immuno” is employed in the present application to refer to any biological analysis using at least one binding partner suitable for binding to the analyte sought, and detecting and/or quantifying the latter, preferably specifically, even when said binding partner is not of immunological nature or origin in the strict sense.

The first binding partner P1 and the second binding partner P2 are chosen, for example, from the group consisting of antibody, mixture of antibodies, antibody fragment, mixture of antibody fragments, antibody analogue, mixture of antibody analogues, antigen, mixture of antigens, protein, mixture of proteins, polypeptide, mixture of polypeptides, peptide, mixture of peptides.

Preferably, the first binding partner P1 is a “capture binding partner”, insofar as it is immobilized, directly or indirectly, on a solid surface. The second binding partner P2 is, preferably, a “detection binding partner” and is associated—directly or indirectly—with a detection element making it possible to reveal the presence of this second binding partner P2, and above all, of any element(s) (analyte(s), reagent(s), etc.) bound to it. This detection element may be a labelling reagent, but more broadly, any element making it possible to reveal the presence of this second binding partner P2, and above all, of any element(s) (analyte(s), reagent(s), etc.) bound to it. By way of illustration, within the framework of the above-mentioned “Magnotech” platform, the nanoparticle associated with the detection binding partner (detection antibody in the present case in question) plays the role of detection element, insofar as the attenuation of the light beam reflected due to this nanoparticle reveals the presence of the detection antibody in the detection region, and consequently, the formation of a sandwich-type complex between the detection antibody, the analyte sought and the capture antibody.

According to a particular embodiment, the detection element is a labelling reagent able to directly or indirectly generate a detectable signal. Purely by way of illustration (non-limiting), these labelling reagents may consist in:
enzymes which produce a signal detectable for example by colorimetry, fluorescence, luminescence, such as horseradish peroxidase, alkaline phosphatase, β-galactosidase, glucose-6-phosphate dehydrogenase,
chromophores such as fluorescent, luminescent and dyeing compounds,
fluorescent molecules such as Alexas or phycocyanins,
radioactive molecules such as ³²P, ³⁵S or ¹²⁵I,
metallic or alloy particles, such as colloidal gold particles,
polymer particles, such as coloured latex particles.

Indirect detection systems may also be used, such as for example ligands able to react with an anti-ligand. Ligand/anti-ligand pairs are well known to the person skilled in the art, which is the case for example with the following pairs: biotin/streptavidin, hapten/antibody, antigen/antibody, peptide/antibody, sugar/lectin, molecule/receptor. In this case, it is the ligand which carries the binding partner. The anti-ligand may be detectable directly by the above-mentioned labelling reagents, or itself be detectable by a ligand/anti-ligand.

These indirect detection systems may lead, under certain conditions, to an amplification of the signal. This signal amplification technique is well known to the person skilled in the art, and reference may notably be made to prior patent applications FR98/10084 or WO-A-95/08000 in the name of the Applicant.

The formation of the sandwich-type complex between the first binding partner P1 and the second binding partner P2 can be revealed by any technique known to the person skilled in the art, notably by directly viewing a reaction due to the labelling reagent associated with the second binding partner P2, or using a detection means, for example optical type, as is the case within the framework of the “Magnotech” operating protocol.

Advantageously, at least one of the first and second binding partners P1 and P2—preferably both—are of immunological nature and/or origin. Preferably, at least one of said binding partners P1 and P2—preferably both—are antibodies. “Antibody” is to be understood to mean a polyclonal antibody, a monoclonal antibody, a humanized antibody, a human antibody or a fragment of said antibodies, in particular the fragments Fab, Fab′, F(ab′)2, ScFv, Fv, Fd. The requisite condition is that said antibodies must be specific to the analyte sought, i.e. wherever possible, they do not have any cross reactions with other analytes; antibodies with the highest affinities and lowest dissociation constants being the antibodies preferred for the purposes of the present invention.

Polyclonal antibodies can be obtained by immunisation of an animal with the appropriate immunogen, followed by the recovery of the antibodies sought in purified form, by sampling the serum of said animal, and separation of said antibodies from the other constituents of the serum, in particular by affinity chromatography on a column on which is immobilized an analyte (antigen) specifically recognized by the antibodies.

Monoclonal antibodies can be obtained by the hybridoma technique, the general principle of which is recapped below.

In a first stage, an animal, generally a mouse, is immunized with the appropriate immunogen, the B lymphocytes of which are then capable of producing antibodies against this antigen. These antibody-producing lymphocytes are then fused with “immortal” myeloma cells (generally murine) to give rise to hybridomas. From the heterogeneous mixture of the cells thus obtained, cells are then selected that are capable of producing a particular antibody and of reproducing indefinitely. Each hybridoma is reproduced in the form of a clone, each leading to the production of a monoclonal antibody, whose recognition properties with respect to the antigen sought will be able to be tested for example via ELISA, by immunotransfer (Western blot) in one or two dimensions, via immunofluorescence, or by means of a biosensor. The monoclonal antibodies thus selected are then purified, for example, by affinity chromatography.

The monoclonal antibodies can also be recombinant antibodies obtained by genetic engineering, by techniques well known to the person skilled in the art.

In the embodiment wherein the first and second binding partners P1 and P2 are antibodies, the analyte sought is referred to as an “antigen”, and the first and second binding partners P1 and P2 (antibodies) recognise two distinct epitopes—respectively known as E1 and E2—of this antigen.

If P1 and P2 are antibodies, P1 is preferably a “capture antibody”, insofar as it is immobilized, directly or indirectly, on a solid surface. The antibody P2 is, preferably, a “detection antibody” and is associated—directly or indirectly—with a detection element making it possible to reveal the presence of this second binding partner P2, and above all, of any/all element(s) (analyte(s), reagent(s), etc.) bound to it. This detection element is as defined above.

The expression “immobilized . . . binding partner” means that the binding partner is fixed directly or indirectly within the device by any method known to the person skilled in the art, such as by adsorption or covalent bonding.

The expression “peptide containing an epitope recognized by an antibody” refers to any sequence of amino acids comprising at least the epitope recognized by the binding partner concerned. The peptide can be limited to the epitope itself, or comprise several additional amino acids, until it reaches the protein, insofar as the peptide retains the recognition activity by the binding partner concerned. The peptide can also contain one or more muted amino acids, insofar as, once more, the peptide retains the recognition activity by the binding partner concerned. Within the framework of a capture partner (also known as a capture binding partner) and detection partner (also known as detection binding partner) pair, it may be preferable for the capture partner control not to contain the detection partner control epitope, and vice versa. Nonetheless if using several partners for the same function (for example two capture partners), the peptide may comprise the first capture partner epitope E1 and the other capture partner epitope E2.

The term “matrix” refers to any type of material capable of ensuring the flow and the transfer of a liquid. The liquid can be transferred by capillary force. This is particularly advantageous if the device according to the invention is a device making it possible to employ a lateral flow assay. The matrix may be, for example, made of at least one bibulous material. Bibulous materials are materials which easily absorb a liquid, and across which the liquid is transported by capillarity. By way of bibulous materials, mention can be made, for example, of nitrocellulose, polyester, glass fibres, etc.

### BRIEF DESCRIPTION OF THE DRAWINGS

The invention, its functionality, its applications and its advantages shall be better understood by reading the present description, made with reference to the following figures, wherein:

FIG. 1 is a schematic representation of the concept underlying the present invention;

FIG. 2 is a top view of a reaction cartridge usable in a “Magnotech” type opto-magnetic immunoassay device;

FIGS. 3A, 3B, 3C and 3D illustrate the operation of an opto-magnetic immunoassay technology platform, such as the “Magnotech” platform;

FIG. 4 is a top view of a device according to a first embodiment of a first aspect of the invention;

FIG. 5 illustrates, schematically, a first configuration of this first embodiment of the invention;

FIG. 6 represents, schematically, a second configuration of this first embodiment of the invention;

FIG. 7 is a top view of a device according to a second embodiment of a first aspect of the invention;

FIG. 8 represents, schematically, the device according to this second embodiment;

FIG. 9 represents, schematically, a third embodiment of a first aspect of the invention;

FIGS. 10A, 10B, 10C and 10D represent top views of a lateral flow test as described in application WO 2012/066235, in the name of the Applicant;

FIGS. 11A, 11B, 11C, 11D, 11E and 11F represent, schematically, top views of a device according to a second aspect of the invention, suitable for employing a unilateral flow assay;

FIG. 12 is a schematic representation of biological control of the binding partner P2 within the framework of detecting troponin I (TnI) as an analyte, P1 and P2 being anti-TnI monoclonal antibodies, respectively 19C7 and 560, and CTRL2 containing an epitopic peptide of P2;

FIG. 13 is a schematic representation of a Magnotech cartridge possessing two reaction chambers, wherein the binding partner P2 is biologically controlled, chamber 1 being coated with two spots, each for detecting troponin I (P1 spot on which the anti-troponin I monoclonal antibody 19C7 (binding partner P1)) is immobilized, and chamber 2 being coated with three spots, one of the spots being for the detection of troponin I (P1 spot, identical to the P1 spots in chamber 1) and the other two (CTRL2-1 spot and CTRL2-2 spot) being for the biological control of the anti-troponin I detection antibodies (monoclonal antibody 560, binding partner P2), using a control CTRL2 (protein coupled to the epitopic peptide of P2);

FIG. 14 sets out the signal results obtained in chambers 1 and 2 of the Magnotech cartridge according to FIG. 13, with three different samples, namely one TnI negative sample, and two TnI positive samples, with two different concentrations of TnI;

FIG. 15 is a schematic representation of biological control of the anti-troponin I monoclonal antibody 19C7 (binding partner P1), using a control CTRL1 (protein coupled to the epitopic peptide of P1 and to a magnetic particle), within the framework of a Magnotech technology used for detecting troponin I as an analyte; and

FIG. 16 sets out the signal results obtained in a chamber presenting a biological control of the binding partner P1 according to FIG. 15, with two different samples, namely one TnI negative sample, and one TnI positive sample.

### DETAILED DESCRIPTION OF THE INVENTION

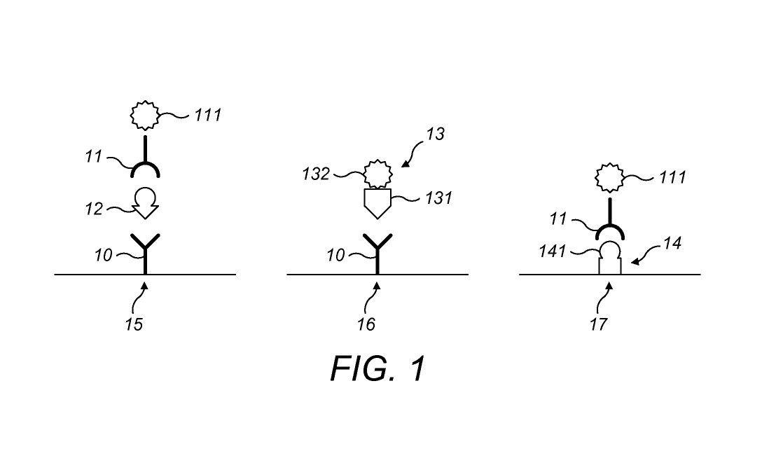

As indicated above, FIG. 1 is aimed at illustrating the concept underlying the present invention. Said concept relates to increasing the detection sensitivity within the framework of any type of sandwich-type immunoassay, through significantly reducing—or even eliminating—false negative results. To this end, we use a first binding partner P1, in this case antibody 10, immobilized within the device according to the invention. This antibody 10 is used as a capture antibody.

A second binding partner P2 is also required, in this case antibody 11, immobilizable by forming a sandwich-type complex with the analyte 12 and capture antibody 10. The antibody 11, for its part, is known as the “detection antibody”. This detection antibody 11 is associated, directly or indirectly, with a detection element 111 making it possible to detect the formation of the “sandwich” between the capture antibody 10, the analyte 12 and detection antibody 11; the formation of said sandwich revealing the presence of the analyte 12 sought within the tested sample (not numbered in FIG. 1). Of course, the type of detection element 111 used depends on the detection means employed. For example, if a fluorescent or coloured reaction is detected, the detection element 111 will consist, respectively, in a fluorogenic or chromogenic element (namely, making it possible to obtain a fluorescent reaction or a coloured reaction), such as an enzyme in the presence of a substrate. If, as indicated above, the sandwich-type immunoassay is employed via an opto-magnetic technology platform such as the “Magnotech” platform, the detection element 111 may simply comprise a magnetic particle/ball. Indeed, this magnetic particle will influence the intensity of the light signal reflected in the detection region, and thus will provide an indication on the formation of the “sandwich”, thus revealing the presence/quantifying or the absence of the analyte 12 sought.

In order to increase detection sensitivity, the inventive concept underlying the present invention requires use of two positive controls:
(i) a first positive control 13 making it possible to test not only for the presence of but above all the functional character of the capture antibody 10. This positive control 13 comprises an epitope 131 (for example of a peptidic nature) specifically recognized by the capture antibody 10 and a detection element 132, identical to or different from the detection element 111;
(ii) a second positive control 14 making it possible to test not only for the presence of, but above all the functional character of the detection antibody 11, said control 14 comprising an epitope 141 (for example of a peptidic nature) specifically recognized by the detection antibody 11.

According to a particular embodiment, epitopes 131 and 141 may be identical, and therefore antibodies 10 and 11 may also be, for example if the analyte possesses several identical epitopes. Preferably, epitopes 131 and 141 are different if the analyte possesses only different epitopes, as is the case, for example, for troponin I.

In the presence of the analyte sought within the tested sample, the operator will detect (visually or using a detection means):
a signal revealing the formation of a sandwich-type complex between the capture antibody 10, the analyte 12 and the detection antibody 11, in the detection and/or quantification region of the analyte 15,
a signal revealing the formation of a complex between the capture antibody 10 and the biological control 13, in the capture antibody biological control region 16, and
a signal revealing the formation of a complex between the detection antibody 11 and the biological control 14, in the detection antibody biological control region 17.

Conversely, if the tested sample does not contain the analyte sought but the test is functional (namely if each of the capture 10 and detection 11 antibodies is operational), the following will be observed:
signal absence in the analyte detection and/or quantification region 15,
a signal revealing the formation of a complex between the capture antibody 10 and the biological control 13, in the capture antibody biological control region 16,
a signal revealing the formation of a complex between the detection antibody 11 and the biological control 14, in the detection antibody biological control region 17.

If a signal absence is obtained in the analyte detection and/or quantification region 15, and a signal absence is also observed in the capture antibody biological control region 16 and/or in the detection antibody biological control region 17, the test result must be considered as uninterpretable. In the absence of biological control of the capture antibody 10, in the biological control region 16, the test result would have been wrongly considered negative, whereas the analyte of interest was actually contained in the tested sample.

Furthermore, it can easily be observed in FIG. 1 that the device according to the invention makes it possible to clearly identify the defective binding partner(s) (in this case the capture antibody 10 and/or the detection antibody 11). This notably represents an advantage in the production of kits comprising a support (plate, reaction cartridge, etc.) on which capture binding partners and a solution comprising the detection antibodies are immobilized (for example by adsorption). Indeed, if when using the concept according to the present invention, it proves that the functional character of the capture antibody 10 has been altered, the support will be considered unusable, and must therefore be destroyed.

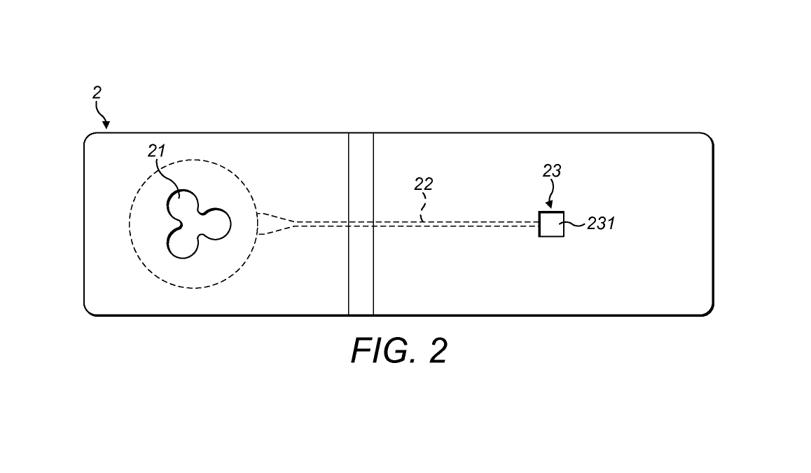

An example of a reaction cartridge 2 used in a “Magnotech” type opto-magnetic immunoassay device is presented in FIG. 2. This reaction cartridge 2 comprises an inlet 21 designed to receive the liquid sample to be tested, a channel 22 enabling the sample to be tested to flow (for example naturally or by capillarity) toward the reaction area 23 comprising, in the example represented in FIG. 2, a single reaction chamber 231.

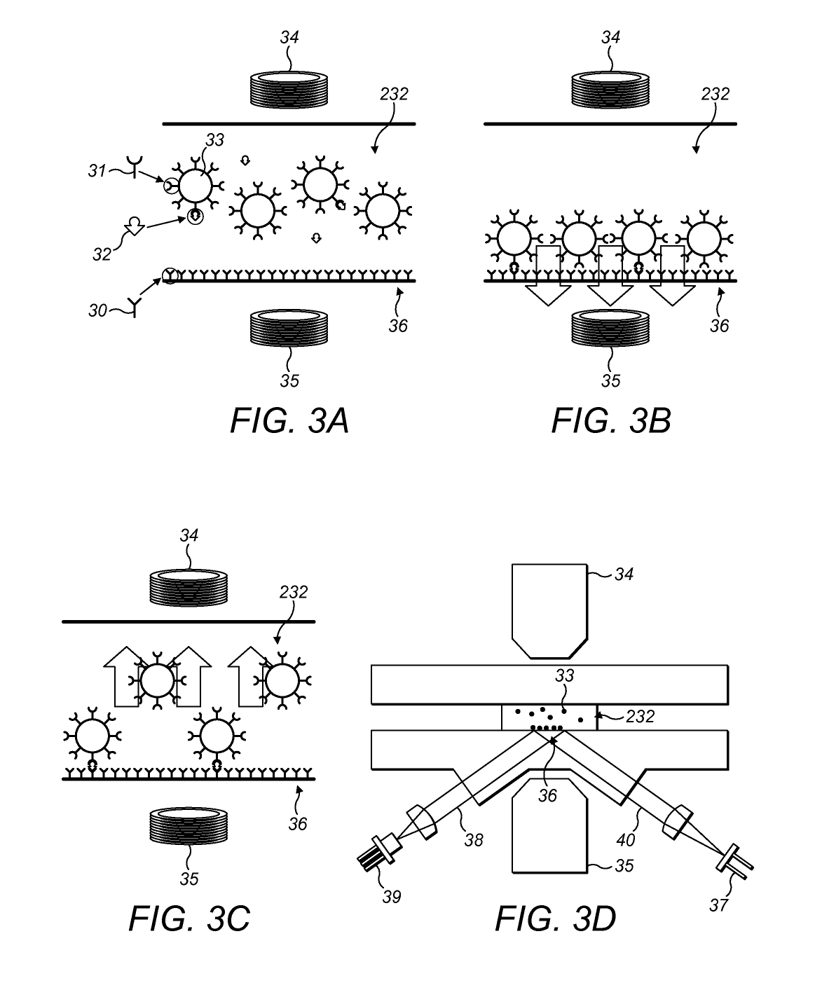

As indicated throughout the present application, the device according to the invention is usable for various types of immunoassay, and in particular in an opto-magnetic immunoassay using the “Magnotech” platform developed by Philips (Eindhoven, Netherlands), the principle of which is recapped below with reference to FIGS. 3A-3D.

In the reaction chamber 231 of the reaction cartridge represented in FIG. 2, the capture and detection antibodies are maintained in dry form. In the first step, represented in FIG. 3A, the magnetic particles 33, coated with detection antibodies 31, are re-suspended in the presence of the liquid sample previously introduced via the inlet 21 of the reaction cartridge 2 represented in FIG. 2.

Then, in a second step, represented in FIG. 3B, the lower electromagnet 35 is activated in order to attract the magnetic particles 33 toward the detection region 36 on which capture antibodies 30 are immobilized (for example by covalent bonding or adsorption), thus enabling, if the analyte 32 sought is present, the formation of a sandwich-type complex between the capture antibody 30, the analyte 32 and the detection antibody 31, carried by a magnetic particle 33.

Finally, in the third step, represented in FIG. 3C, the lower electromagnet 35 is deactivated and the upper electromagnet 34 is activated in order to induce the withdrawal of the magnetic particles 33 not bound to the detection region 36—and thus retain only, in said detection region 36, the magnetic particles 33 of which at least one detection antibody 31 is bound to at least one capture antibody 30 by means of at least one analyte 32 sought (formation of a sandwich-type complex between the three above-mentioned entities), if the analyte of interest is present in the tested sample.

In light of the above, the lower electromagnet 35 can be considered a “binding” electromagnet, whereas the upper electromagnet 34 can be considered a “washing” electromagnet.

Possibly after steps 2 and 3, represented in FIG. 3C, the magnetic particles/balls present in the detection region 36 are detected using, as a detection means 37, a 2-dimensional photographic sensor (“2D”, such as a CCD sensor), the detection element comprising the magnetic particle 33.

In substance, the detection step, represented in FIG. 3D, is based on the principle of frustrated total internal reflection (f-TIR), as described, for example, in Bruls et al. [1] or in patent application WO2013/054230. Broadly speaking, the detection principle is as follows: an incident light beam 38 is emitted (for example by an LED type light source 39) toward the detection region 36, with an incident angle greater than the critical angle (depending on the constituent material of the reaction cartridge), so that the incident light beam 38 is reflected in full as a reflected light beam 40—known in this case as an “evanescent beam”—toward the detection means 37.

As indicated above, and in summary, the detection principle is based on the following premise: the intensity of the reflected light beam 40 is inversely proportional to the quantity of magnetic particles 33 present in the detection region 36. In other words, the decreased intensity of the reflected light beam 40 observed in the detection means 37 is proportional to the number of magnetic particles 33 bound to the detection surface 36, and consequently, to the concentration of analyte 32 present in the tested sample.

According to a first aspect of the invention, the device according to the invention is suitable for being employed in a “Magnotech” type opto-magnetic immunoassay.

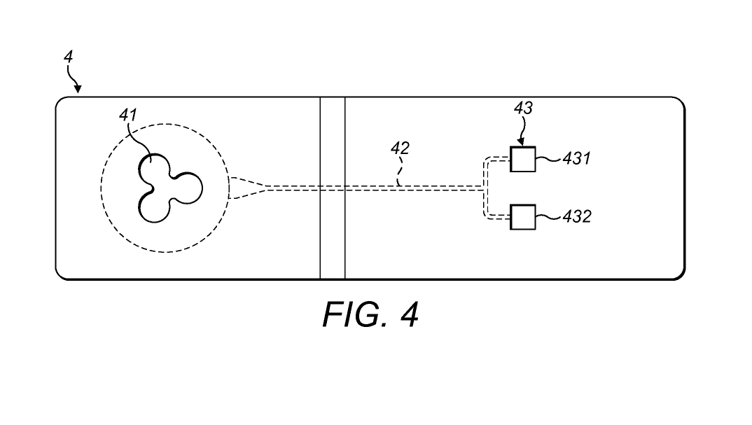

A device according to a first embodiment of the first aspect of the invention is represented in FIG. 4 (top view). This device is a reaction cartridge similar to that represented in FIG. 2. It comprises an inlet 41, a channel 42 and a reaction area 43, with the exception that the reaction area comprises two reaction chambers 431 and 432, which are not in direct fluid communication.

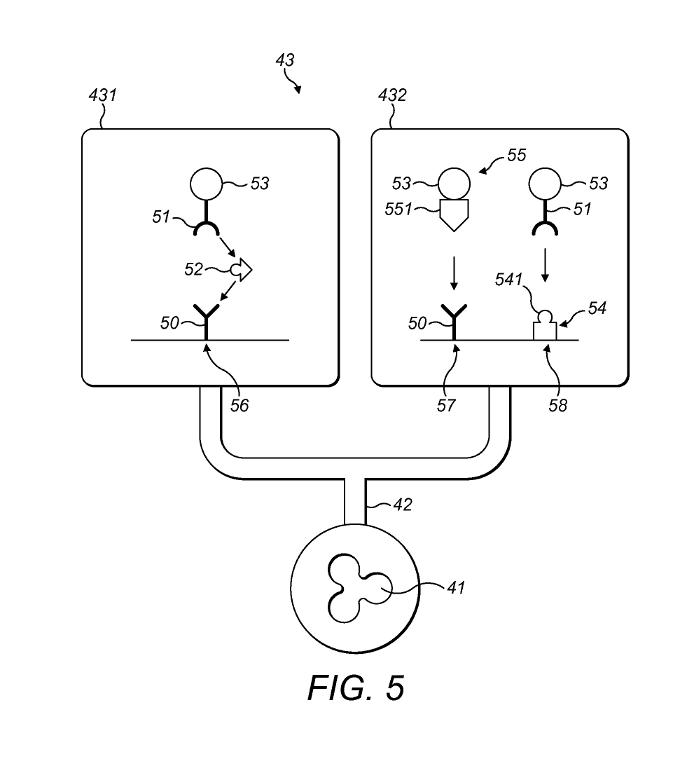

FIG. 5 illustrates, schematically, a first configuration of this first embodiment of the invention during the first step of the “Magnotech” operating protocol, as represented in FIG. 3A.

The first reaction chamber 431 comprises an analyte detection and/or quantification region 56, in which the capture antibody 50 is immobilized. The formation of a sandwich-type complex between the detection antibody 51 (associated with the magnetic particle 53), the analyte 52 and the capture antibody 50 will be revealed in said detection region 56 by attenuation of the reflected light signal 40 in the detection means 37, as shown schematically in FIG. 3D.

According to this first configuration, the second reaction chamber 432 comprises a biological control region of the capture antibody 50, in which, if the capture antibody 50 is functional, the formation of a complex is detected between the epitope 551 of the biological control 55 (associated with the magnetic particle 53) and the capture antibody 50. As described above, the detection, in the capture antibody biological control region 57, is performed by attenuating the light beam 40 in the detection means 37, as shown schematically in FIG. 3D.

This second reaction chamber 432 also comprises a detection antibody biological control region 58, in which, if said detection antibody 51 is operational, a complex will form between said detection antibody 51 (bound to the magnetic particle 53) and the epitope 541 of the biological control 54. Here too, the presence of this complex is revealed, in the detection antibody biological control region 58, by attenuating the light beam 40 in the detection means 37, as shown schematically in FIG. 3D.

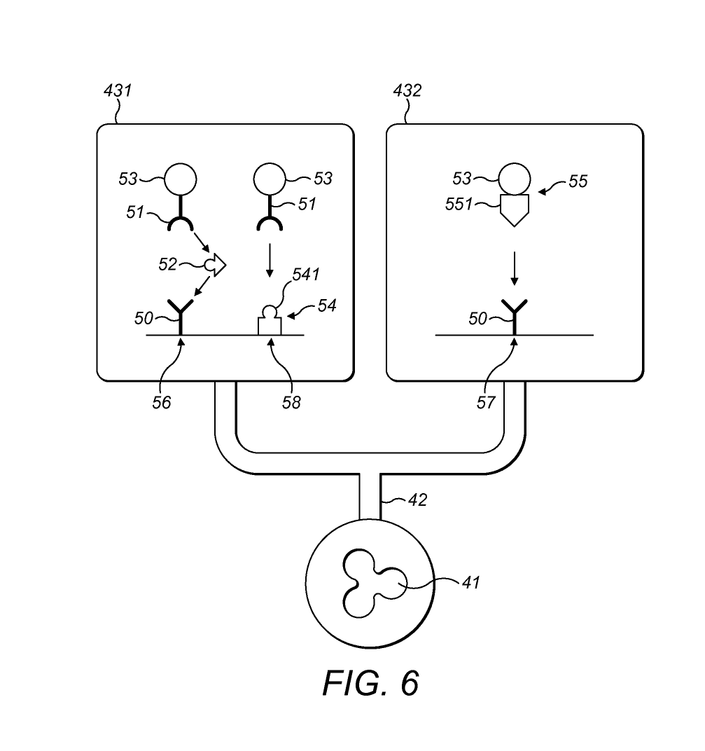

FIG. 6 represents a second configuration of the first embodiment according to a first aspect of the invention. Just like the first configuration represented in FIG. 5, the one shown in FIG. 6 comprises two reaction chambers 431 and 432. However, according to the configuration represented in FIG. 6, the reaction chamber 431 comprises both the analyte detection and/or quantification region 56 and the detection antibody biological control region 58. The reaction chamber 432, for its part, comprises the capture antibody biological control region 57.

Thus, by placing the capture antibody biological control region 57 in a reaction chamber distinct from the chamber 431, comprising the analyte detection and/or quantification region 56 and the antibody biological control region 58, a competition phenomenon is prevented between the analyte 52 and biological control 55 with respect to binding to the capture antibody 50 in the analyte detection and/or quantification region 56.

In the second configuration shown schematically in FIG. 6, it is important, or even essential, that the detection antibody 51 is present in excess in relation to the number of binding sites constituted by the analyte 52 and epitope 541 of the biological control 54, in order to prevent a competition phenomenon with respect to binding of said detection antibody 51 to the analyte 52 or to the biological control 54.

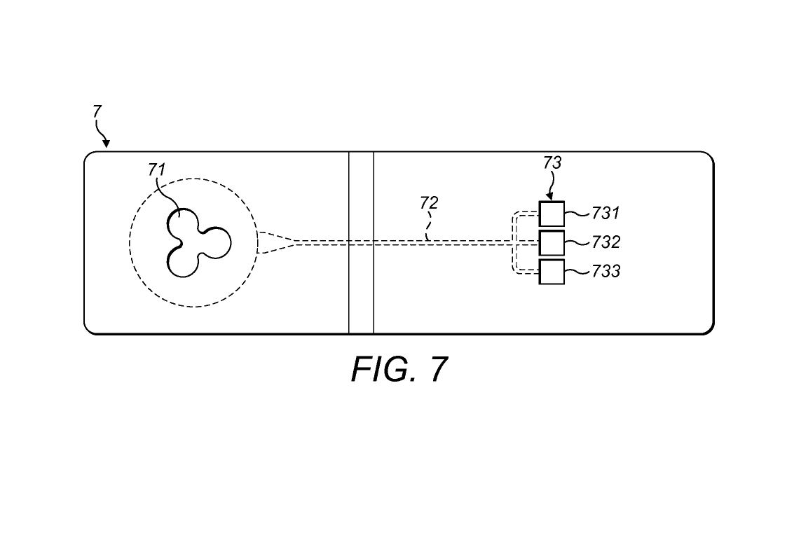

FIG. 7 is a top view of a device according to a second embodiment of the first aspect of the invention. More specifically, this FIG. 7 represents a device according to the invention, in the form of a reaction cartridge, usable in a “Magnotech” type opto-magnetic device. This reaction cartridge is similar to that represented in FIG. 4, with the exception that the reaction area 73 does not comprise two distinct reaction chambers, but three distinct reaction chambers 731, 732 and 733, which are not in direct fluid communication.

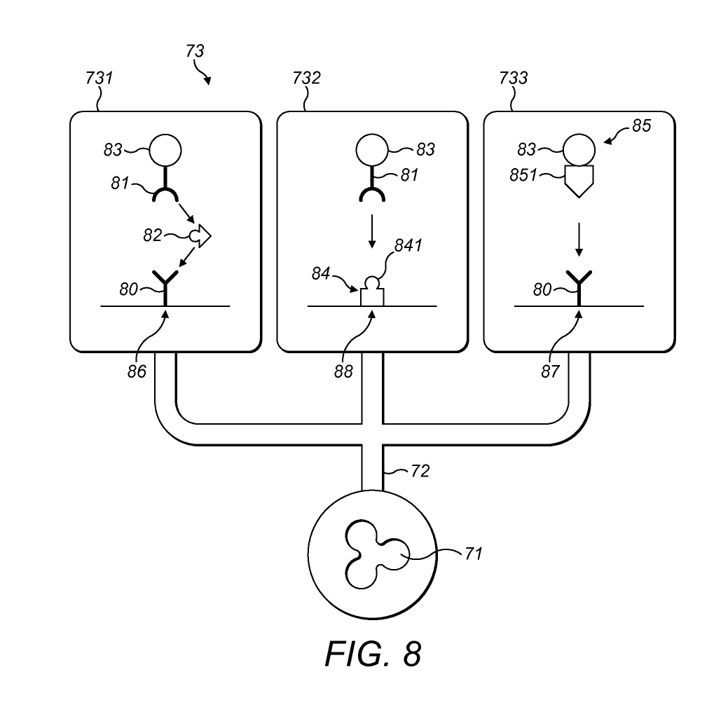

These three reaction chambers 731, 732 and 733 are represented, schematically, in FIG. 8.

The first reaction chamber 731 comprises the analyte detection and/or quantification region 86, in which the complex forms between the capture antibody 80, the analyte 82 and the detection antibody 81, provided that the analyte 82 is present in the tested sample.

The reaction chamber 732 comprises the detection antibody biological control region 88, in which the complex forms between the detection antibody 81, bound to the magnetic particle 83, with the epitope 841 of the biological control 84, provided that the detection antibody 81 is functional.

The third reaction chamber 733 comprises the capture antibody biological control region 87, in which a complex forms between the epitope 851, bound to the magnetic particle 83, of the biological control 85 and the capture antibody 80, provided that the latter is operational/functional.

The presence of these complexes in their respective detection areas 86, 88 and 87 is detected as indicated above with reference to FIG. 3D.

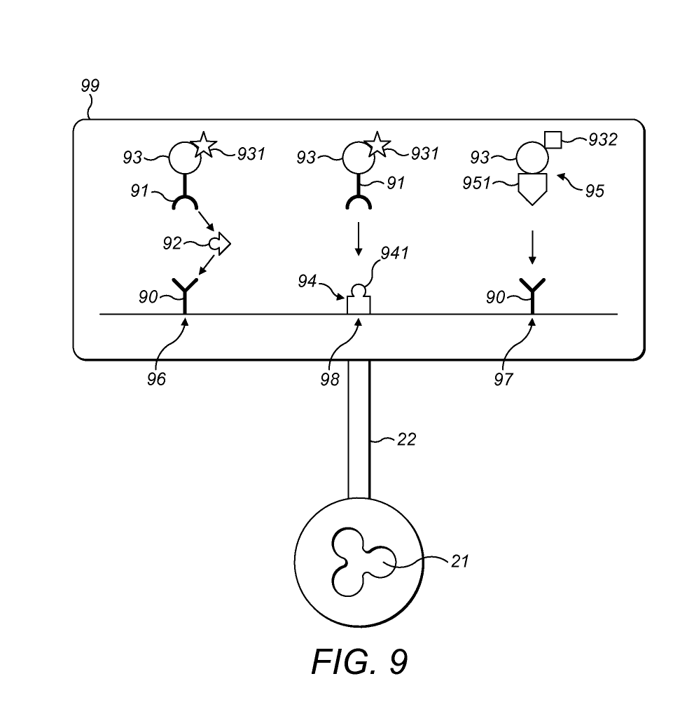

A third embodiment according to a first aspect of the invention is shown schematically in FIG. 9. The latter concerns a device according to the invention in the form of a reaction cartridge comprising a single reaction chamber, as shown in FIG. 2.

In this third embodiment, the sole reaction chamber 99 comprises:
an analyte detection and/or quantification region 96, in which a complex forms between the capture antibody 90, the analyte 92 and the detection antibody 91 (associated with the magnetic particle 93) in the presence of the analyte 92 sought;
a detection antibody biological control region 98, in which a complex forms between the detection antibody 91, bound to the magnetic particle 93, and the epitope 941 of the biological control 94, if the latter is functional;
a capture antibody biological control region 97, in which a complex forms between the latter and the epitope 951, bound to the magnetic particle 93, of the biological control 95 (bound to a magnetic particle 93), if said capture antibody 90 is functional.

As stated with reference to FIG. 6, it is important for the detection antibody 91 to be present in excess in relation to its possible binding sites (constituted by the analyte 92 and the biological control 94) in order to prevent the competition phenomenon presented above. Furthermore, in this third embodiment, it is also important for the capture antibody 90 to be present in excess in relation to its possible binding sites (namely the antigen 92 and the epitope 951 of the biological control 95), in order to prevent specific binding of the epitope 951 of the biological control 95 to the capture antibody 90, in the detection and/or quantification region 96, from impeding the formation of a sandwich-type complex in said detection and/or quantification region 96. It is also important for the epitope 951 of the biological control not to be present in excessive quantity for the same reason. This is particularly important if the analyte is present in low quantity in the sample to be assayed.

According to this embodiment, and insofar as it is possible for the epitope 951 of the biological control 95 to bind not only to the capture antibody 90 in the capture antibody biological control region 97, but also in the analyte detection and/or quantification region 96, it is important or even essential for the magnetic particles 93 associated with the biological control 95 to be associated, directly or indirectly, with a label 932 making it possible to detect which complexes, in regions 96 and 97, are due to the binding of the biological control 95 with the capture antibody 90, and thus indirectly deduce therefrom which complexes actually reveal the presence of the analyte 92 in the tested sample.

The various regions may also be differentiated using different coloured particles or particles of different diameters.

According to one alternative, only the magnetic particles 93 associated with the detection antibodies 91 are labelled with a label 931, in order to directly identify the sandwich-type complexes in the analyte detection and/or quantification region 96.

According to one particular embodiment, the magnetic particles 93 associated with the biological control 95 and the magnetic particles 93 associated with the detection antibodies 91 are respectively labelled with a label 932 and 931, said labels 932 and 931 being different from each other.

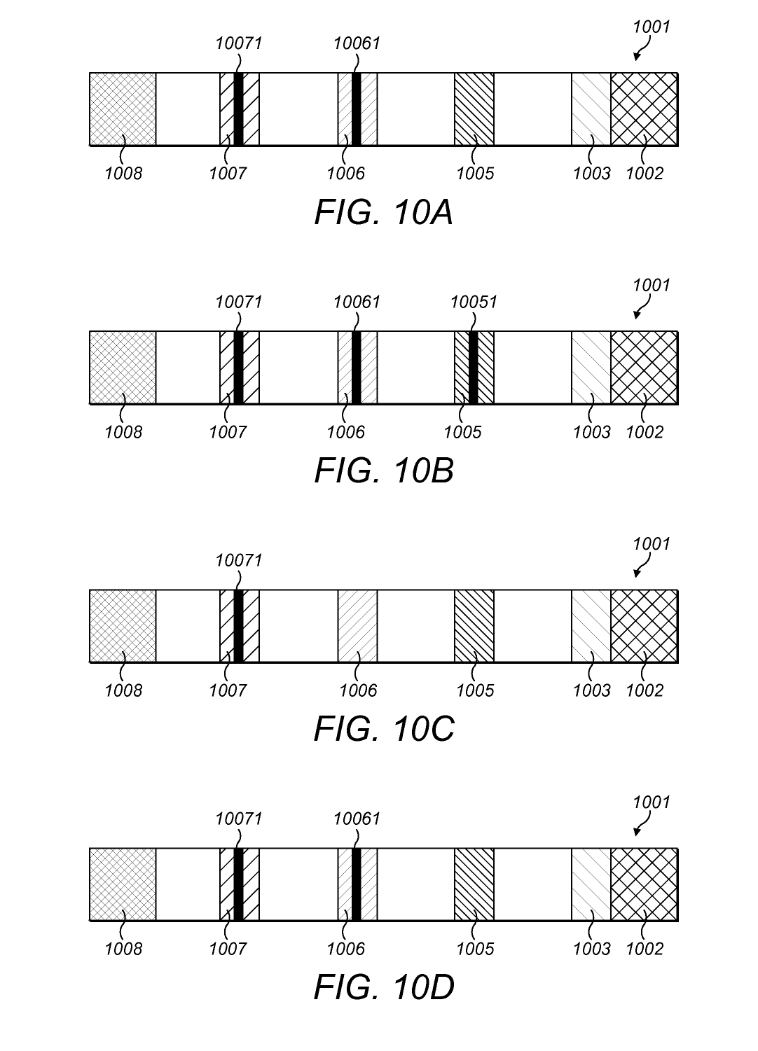

As indicated above, the present invention finds applications in various types of sandwich-type immunoassay, and notably in rapid unit tests, also known as lateral flow tests/assays. As stated above, patent application WO 2012/066235, in the name of the Applicant, discloses a device making it possible to perform a rapid unit test, said device incorporating a positive control. The operation of this prior art device is illustrated in FIGS. 10A-10D.

In summary, the device according to WO 2012/066235 is a cassette comprising a support (not represented), a matrix 1001 comprising a liquid sample application area 1002, a labelling area 1003, a results viewing area 1005, a positive control area 1006, an optional migration control area 1007 and, optionally, a sample absorption area 1008.

As represented in FIGS. 10A-10D, the matrix 1001 is represented in the form of a rectangular strip, the longitudinal axis of which is in the horizontal position. Areas 1002, 1003, 1005, 1006, 1007 and 1008 are in fluid communication. Area 1003 comprises a visibly labelled detection partner (for example a particle of coloured latex, a gold particle, etc.). This labelled antibody can migrate freely across the matrix 1001 and react with the analyte sought (antigen in the present case), if the latter is present in the tested sample. In the results viewing area 1005 of the matrix 1001, a capture antibody having a specificity for an epitope of the antigen different from that recognized by the detection antibody, is immobilized (for example by covalent bonding or adsorption). In the positive control area 1006 of the matrix 1001, an antigen analogue is either immobilized directly or indirectly, or it can migrate freely in the presence of fluid flow in area 1006 until it is immobilized by the capture antibody in the positive control area 1006.

FIGS. 10A, 10B and 10C summarize the operation of the test according to patent application WO2012/066235.

FIG. 10D, comparable at first glance to FIG. 10A, also illustrates the representation of a false negative result obtained using the above-mentioned cassette as explained below.

More specifically, if the sample is a negative control (namely that the analyte sought is not present within said sample), and as illustrated in FIG. 10A, there are no detectable signal emissions in the results viewing area 1005. Conversely, the presence of a signal 10061 (“positive control line”) can be detected in the positive control region 1006, as well as in the migration control region 1007 (signal 10071; also known as “migration control line”). According to patent application WO 2012/066235, this means on the one hand that the negative control sample has migrated to area 1006, and on the other hand that the device is functional. The functionality of the device is deduced from the functional character of the detection antibody, revealed by signal 10061 (“positive control line”) in the positive control region 1006. In other words, the test is such that it indicates that the sample does not comprise any analyte, and is considered uninterpretable according to patent application WO2012/066235.

FIG. 10B illustrates the result obtained with a positive control sample. As represented in this FIG. 10B, the emission of a signal 10051 (“test line”) is detectable in the results viewing area 1005. This detectable signal 10051 is due to the formation of a sandwich-type complex between the capture antibody, the antigen and the detection antibody, in the results viewing area 1005. The detectable signal 10051, strictly speaking, is due to the label associated with the detection antibody. There is also an emission of a detectable signal 10061 in the positive control area 1006, patent application WO 2012/066235 concluding that this means on the one hand that the sample has migrated, and on the other hand that the device is functional. In other words, the test is such that it indicates that the sample comprises the analyte, and is considered interpretable according to patent application WO2012/066235.

FIG. 10C illustrates a result regarded as “uninterpretable” insofar as no signal is detected either in the results viewing area 1005, or in the positive control region 1006.

By multiplying the tests with positive control samples (namely comprising the analyte sought), the Applicant, surprisingly, updated in very small proportions the profile represented in FIG. 10D, identical to the one depicted in FIG. 10A, namely not presenting any signals in the results viewing area 1005 but with a signal 10061 in the positive control area 1006 and a signal 10071 in the migration control area 1007.

Insofar as the positive control sample used in the performance of this test comprises, by definition, the analyte sought, the Applicant therefore concluded that, drawing on the lessons of application WO2012/066235, it was still possible to obtain false negative results. Against all expectation, the Applicant revealed that by adding a capture antibody control to the cassette, the latter presented a detection sensitivity greater than the cassette described in application WO 2012/066235.

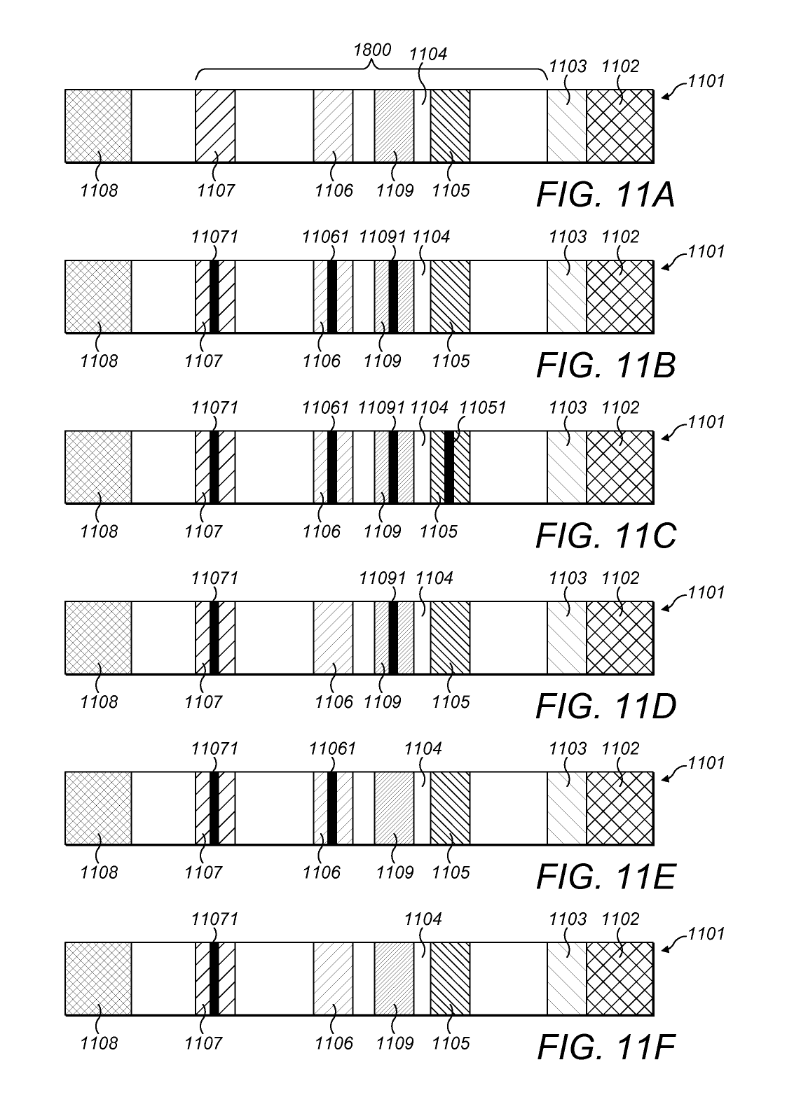

FIGS. 11A, 11B, 11C, 11D, 11E and 11F illustrate the operation of a cassette similar to that described in WO2012/066235 but with increased detection sensitivity. FIGS. 11A-11F represent top views of a lateral flow assay device according to a second aspect of the invention.

As represented in FIG. 11A, this device comprises a support (not represented), a matrix 1101 (represented in the form of a rectangular strip, the longitudinal axis of which is in the horizontal position), said matrix comprising a sample application area 1102, a labelling area 1103 and a reaction area 1800. This reaction area comprises an analyte detection region (in this case an antigen) 1105, a capture binding partner positive control region 1109 (for example an antibody), a detection binding partner positive control region 1106 (for example an antibody) and, optionally, a migration control region 1107. The matrix 1101 can also comprise, optionally, a sample absorption area 1108.

To prevent the competition phenomenon between the analyte of the sample to be assayed and the first analyte analogue CTRL1 in the analyte detection region 1105, the first analyte analogue CTRL1 (consisting for example in an epitopic peptide of P1 coupled to a label or a labelling precursor) is placed downstream (in the direction of liquid migration) of this analyte detection region 1105, but upstream of the capture antibody positive control line in which P1 is immobilized, namely either in region 1104 (situated between regions 1105 and 1109), or in a sub-region of the capture antibody positive control region 1109, situated upstream of said capture antibody positive control line, in which the capture antibody P1 is immobilized.

The detection antibody P2 positive control region 1106, for its part, comprises the second analyte analogue CTRL2 immobilized in the detection antibody P2 positive control line. The labelling area 1103 comprises the detection antibody P2 in excess.

The capture partner P1 positive control region 1109, where applicable comprising said sub-region upstream of said capture antibody positive control line, as indicated above, can also be found after the detection partner P2 positive control region 1106 (not shown). The reaction area 1800 therefore comprises, in order, an analyte detection region (in this case an antigen) 1105, a detection antibody positive control region 1106, a capture antibody positive control region 1109 and, optionally, a migration control region 1107. The matrix 1101 can also comprise, optionally, a sample absorption area 1108.

How the various components are included in the device, by immobilization or otherwise, is known to the person skilled in the art and is described, for example, in patent application WO2012/066235.

FIG. 11B illustrates the results obtained with the device according to the invention after application of a so-called “negative control” sample, namely not containing the analyte sought. As the sample is a negative control, there are, by definition, no emissions of a detectable signal in the analyte detection region 1105 (more specifically in the test line). Conversely, there is an emission of a detectable signal in the capture antibody positive control region 1109 (signal 11091 in the capture antibody positive control line), in the detection antibody positive control region 1106 (signal 11061 in the detection antibody positive control line) and in the migration control area 1107 (signal 11071 in the migration control line), if the latter is present. This means on the one hand that the negative control sample has migrated at least as far as the area 1106, or even as far as the migration area 1107 (if the latter is present) and on the other hand, that both the capture antibody and the detection antibody are present and functional.

FIG. 11C illustrates the results obtained with the device according to the invention after application of a sample positive for a predetermined analyte. As illustrated in FIG. 11C, since the sample is positive for the predetermined analyte (antigen), there is an emission of a detectable signal in the analyte detection region 1105 (signal 11051 in the test line), in the capture antibody positive control region 1109 (signal 11091 in the capture antibody positive control line), in the detection antibody positive control region 1106 (signal 11061 in the detection antibody positive control line), as well as in the migration area 1107 (signal 11071), if the latter is present. This means on the one hand that the sample has migrated at least as far as area 1106, or even as far as area 1107 if the latter is present, and that the capture antibody and the binding antibody are both present and functional.

FIGS. 11D, 11E and 11F illustrate results obtained after application of a sample which is apparently negative for an analyte to be determined.

As represented in FIG. 11D, the presence of a signal 11091 in the capture antibody positive control region 1109 indicates that the latter is functional. Conversely, the absence of a signal in the detection antibody positive control region 1106 indicates that the functional character of this detection antibody is at least partially altered. It cannot be concluded that the sample is negative. It can just be concluded that the test is uninterpretable.

Concerning the results illustrated in FIG. 11E, this is the reverse scenario, but comparable. Indeed, although the presence of a detectable signal 11061 in the detection antibody positive control region 1106 indicates the functional character of this detection antibody, the absence of a detectable signal in the capture antibody positive control region 1109 indicates that the latter is at least partially altered. In relation to the prior art device represented in FIG. 10D, the presence of a capture antibody positive control region 1109 makes it possible to identify the result obtained as being potentially a false negative, rather than as seeming to indicate an absence of analyte in the tested sample. As stated above, this makes it possible to drastically increase the sensitivity of this lateral flow assay.

Finally, as represented in FIG. 11F, not detecting any signal in the capture antibody positive control area 1109 and the detection antibody positive control area 1106 indicates that the functional character of both types of antibody is at least partially altered, the test result, of course, being uninterpretable.

According to another embodiment (not shown), the device may comprise two segments S1 and S2, each comprising a sample application area. The two segments, physically separated, are defined below:
the first segment S1 comprises, besides the sample application area, a labelling area and a reaction area comprising an analyte detection region and a detection binding partner P2 positive control region, and
the second segment S2 comprises, besides the sample application area, the capture binding partner P1 positive control region, the latter comprising a sub-region in which the capture partner P1 is immobilized (for example in the form of a P1 positive control line), with the control CTRL1 placed upstream (in the direction of liquid migration) of said sub-region, as explained above.

This embodiment proves particularly beneficial since the user can only use this second segment S2 if the reading of the first segment initially indicates the absence of analyte in the tested sample, but the detection binding partner P2 control CTRL2 is positive. In such a case, the user does not know whether the result obtained is a true negative, namely whether the analyte sought is absent from the tested sample, or whether the capture binding partner P1 is defective. The user can easily decide between these two hypotheses thanks to the second segment S2 (for example by visually or optically reading the latter). Furthermore, this enables the sample of the second segment S2 to be replaced with an appropriate buffer which is interchangeable with the sample in this segment.

The present invention will be better understood using the following examples given by way of illustration, though non-limiting.

### Example 1

### Preparation of Epitopic Peptides for Biological Control of the Binding Partners

1.1. Peptide Synthesis

Peptides containing the epitopes recognized by the anti-troponin I antibodies 19C7 (SASRKLQLK) and 560 (ELTGLGFAELQ) (Hytest, Turku, Finland) were produced by chemical synthesis according to procedures well known to the person skilled in the art, such as the solid-phase peptidic synthesis described by Merrifield, 1962 (Merrifield, 1962, J. Am. Chem. Soc. 85:2149) and Fields et al, 1990 (Fields G B, Noble R L., 1990, Int J Pept Protein Res., 35(3):161-214), using a polystyrene-type polymer containing 0.1-1.0 mMol amines/g of polymer. After chemical synthesis, the peptides are deprotected and cleaved from the polymer in the presence of a mixture of trifluoroacetic acid-ethanedithiol-triisopropylsilane-water (94/2.5/1/2.5 V/V/V/V) for approximately 2 hours. After elimination of the polymer, the peptides are extracted by precipitation in diethyl ether at 0° C. They are purified by techniques such as high-performance liquid chromatography. Lyophilization of the appropriate purification fractions leads to a homogeneous peptide which is characterized by standard physicochemical techniques such as mass spectrometry, high-performance liquid chromatography, and amino acid analysis.

1.2. Coupling the Epitopic Peptides to Bovine Serum Albumin

Each epitopic peptide is coupled to bovine serum albumin (BSA, Proliant, Ankeny, Iowa, USA) by means of sulfo-SMCC (25 mg/ml, Thermo Fischer Scientific Inc., Rockford, Ill., USA). This chemical modification is performed with a BSA/SMCC ratio of 1/10 for an incubation of 1 h at 30° C.+/−1° C., and is followed by a dialysis against a 50 mM PO4, 150 mM NaCl buffer pH 6.8. Each epitopic peptide diluted to 5 g/L in a 50 mM PO4, 150 mM NaCl, 5 mM EDTA buffer pH 6.8 is then placed in contact with the BSA-SMMC in a ratio of 3 molecules of epitopic peptide per molecule of BSA. After 18 h of incubation at 2-8° C., the coupling reaction is blocked by adding 2-aminoethanethiol (2MEA, Thermo Fischer Scientific Inc., Rockford, Ill., USA) at a concentration complying with equimolarity between the molecules of 2MEA and SMCC, and 20 minutes of incubation under agitation. Each BSA-epitopic peptide control is dialysed against PBS, 0.9 g/L azide buffer and kept at 2-8° C.

1.3. Coupling the Epitopic Peptides to Magnetic Particles

Super-paramagnetic particles 500 nm in diameter (Ademtech, Pessac, France) are functionalized with carboxylic groups as described by Jarrige V et al., 2011 (5) and Dittmer et al., 2010 (3). The control—BSA coupled to the epitopic peptide of the monoclonal antibody 19C7—is fixed onto the magnetic particles at respective concentrations of 20 μg/BSA-epitopic peptide per mg of magnetic particles.

### Example 2

### Biological Control of the Binding Partner P2 with the Epitopic Peptide CTRL2

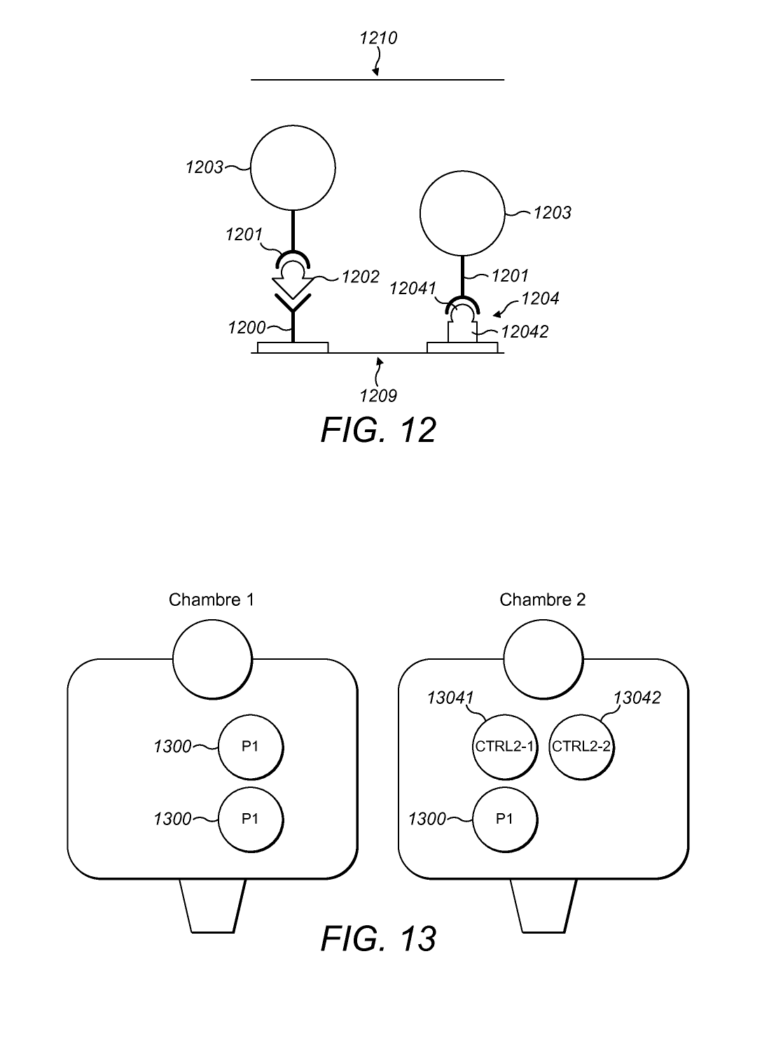

The principle of this control in a test to detect troponin I as the analyte is shown in FIG. 12, P1 1200 and P2 1201 being anti-TnI monoclonal antibodies, respectively 19C7 and 560, P2 1201 being coupled to a magnetic particle 1203 as described above and CTRL2 1204 being a P2 epitopic peptide 12041 coupled to BSA 12042, as described above.

2.1. Preparing the Cartridge

The Magnotech cartridge (Philips, Eindhoven, Netherlands) comprises three elements, namely: the optical part 1209, a strip 1210 and a filtration unit onto which the sample is deposited (not represented).

The optical part is the lower part of the cartridge. It comprises the filtered sample inlet point, connected to two reaction chambers (chamber 1 and chamber 2) via a micro-fluidic channel.

The strip is a biocompatible adhesive film which constitutes the upper part of the cartridge, and closes off the reaction chambers and fluidic channels etched into the optical part.

As shown in FIG. 13, the anti-troponin I antibody 19C7 (binding partner P1) is deposited in spot form into each reaction chamber (P1 spot 1300) by printing (sciFLEXARRAYER, S11, Scienion AG), as described in the article by Dittmer et al. [7] (Journal of Immunological methods, 338: 40-46), at a concentration of 40 μg/mL of antibody.

The control CTRL2 1204 (BSA coupled to the epitopic peptide of the anti-troponin I antibody 560), prepared in example 1.2. and used as a biological control for the binding partner P2 (anti-troponin I antibody 560 coupled to functionalized magnetic particles as described in example 1.3., with the exception that the concentration of antibodies is 60 μg of antibodies per mg of magnetic particles), is also deposited in spot form at a concentration of 0.1 μg/mL of CTRL2 (CTRL2-1 spot 13041) or 1 μg/mL of CTRL2 (CTRL2-2 spot 13042).

The binding partner P2 is deposited onto the surface of the strip 1209 of each reaction chamber by means of the Nanodrop NS-2Stage (Innovadyne Technologies, Inc. Carnforth, UK).

The three elements are then assembled and kept in the presence of a desiccant at 4° C.

2.2. Assaying the Samples

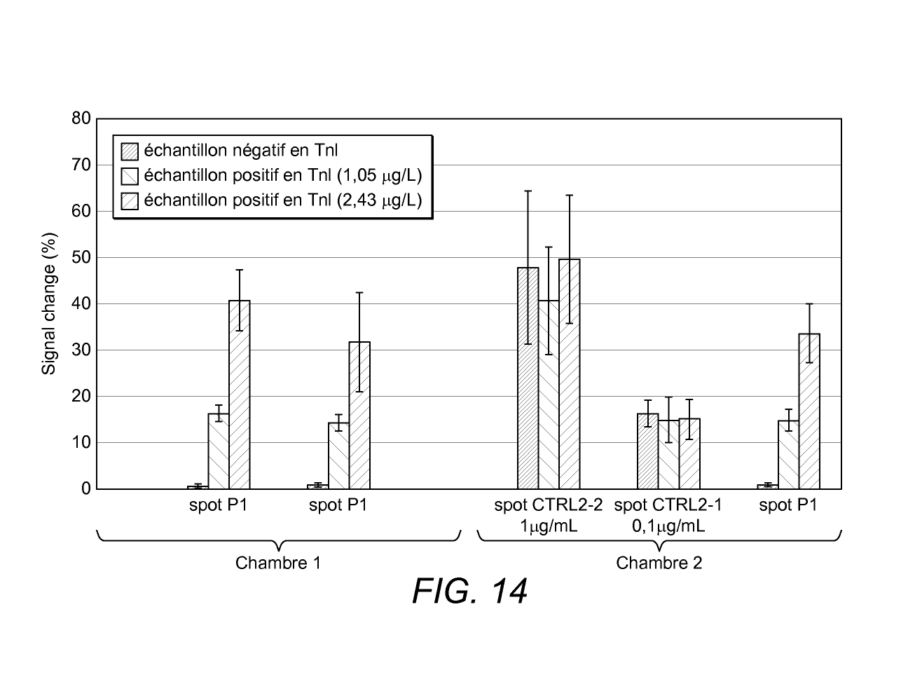

Three types of sample are assayed 10 times by Magnotech technology (Philips, Eindhoven. Netherlands), namely one so-called TnI-negative sample, comprising heparinized plasma, with troponin I concentrations of less than 0.01 and two TnI-positive samples, also prepared with the same heparinized plasma, but overloaded with purified human cardiac troponin (ITC complex, Hytest, Turku, Finland) to achieve concentrations of 1.05 μg/mL and 2.43 μg/mL. The results are averaged and expressed in arbitrary so-called “Signal Change” units, representing the difference between a signal without fixed particles in the detection area (i.e. with no analytes), and a signal with fixed particles (i.e. in the presence of analytes). This “signal change” is expressed in percent. The results are set out in FIG. 14.

The graphs in FIG. 14 show that:
during the assay of the troponin-negative sample, a signal considered negative (less than 0.4%) was observed on the P1 spots 1300 in both chambers, whereas the biological control of the binding partner P2 gave a positive signal (CTRL2-1 spot 13041 and CTRL2-2 spot 13042). This confirms that the antibodies 560 coupled to the balls are functional, and that the sample is a true negative. In addition, it also shows that it is possible to perform the positive control equally well in a chamber distinct from the chamber where analyte detection is performed, or in the same chamber.
the assay of troponin-positive samples with respective concentrations of 1.05 μg/mL and 2.43 μg/mL show an increasing signal at the P1 spots 1300 (signals from 14% to 41%). It is also observed that at the CTRL2 spots 13041 and 13042, no significant modification between the signals obtained with the troponin I-positive samples and those originating from the negative sample was detected. This also shows that the troponin I present in the sample does not affect the biological control, since fixation is observed of the magnetic balls coupled to the anti-troponin I antibody 560 (P2) on CTRL2 (CTRL2 spots 13041 and 13042), even at a high concentration of troponin I.

### Example 3

### Biological Control for the Binding Partner P1 with the Epitopic Peptide CTRL1

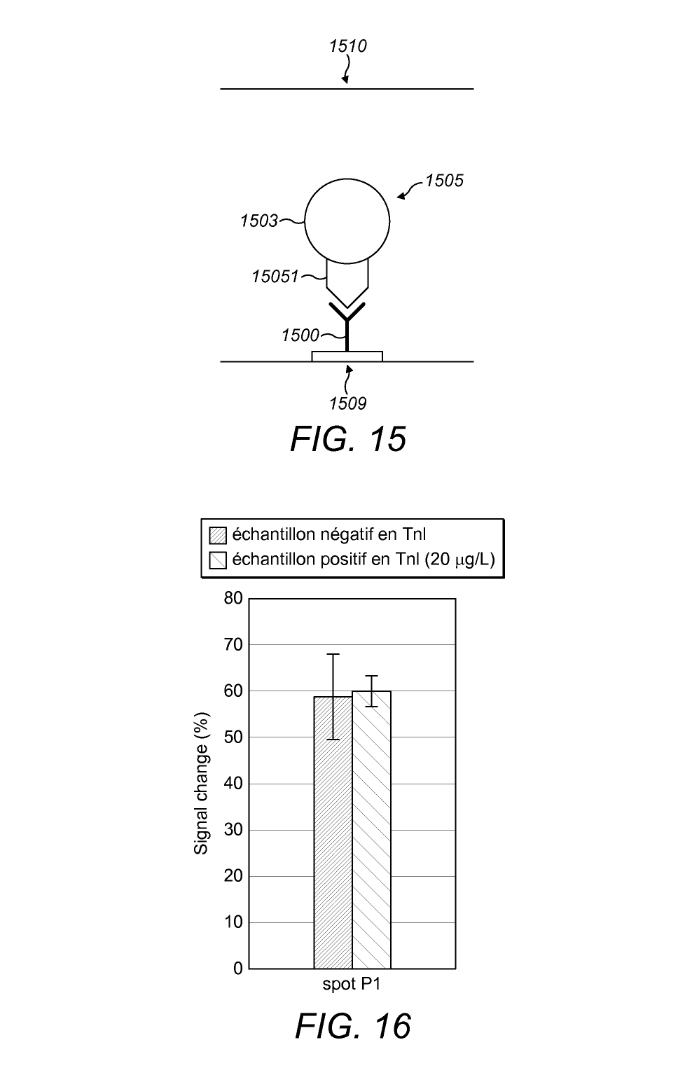

The principle of this control in a test to detect troponin I as the analyte is shown in FIG. 15, P1 1500 being an anti-TnI monoclonal antibody 19C7, and CTRL1 1505 being a P1 epitopic peptide 15051 coupled to magnetic balls 1503, as described above.

3.1. Preparing the Cartridge

The cartridge is prepared as described in point 2.1. above, with the exception that the control CTRL1 1505 prepared in point 1.3. above (magnetic ball 1503 coupled to the epitopic peptide of the anti-troponin I monoclonal antibody 19C7 15051) is deposited onto the strip 1510 in the reaction chamber, opposite the binding partner P1 1500 (monoclonal antibody 19C7 fixed onto the optical part (P1 spot; not represented)).

3.2. Assaying the Samples

Two types of sample are assayed 5 times by Magnotech technology (Philips, Eindhoven, Netherlands), namely one so-called TnI-negative sample, comprising heparinized plasma, with troponin I concentrations of less than 0.01 μg/L, and one TnI positive sample, also prepared with the same heparinized plasma, but overloaded with purified human cardiac troponin (ITC complex) to achieve a concentration of 20 μg/L. The results are the mean, and are expressed as Signal Change (%). They are set out in FIG. 16.

The graphs in FIG. 16 show that:
during the assay of the troponin I-negative sample, a mean positive signal of 59% was obtained (CTRL1 spot; not represented), which demonstrates that the binding partner P1 1500 (antibody 19C7) is functional;
during the assay of the troponin I-positive sample, a mean positive signal of 60% was obtained. This signal level, similar to that of the negative sample, shows the biological control of the binding partner P1 1500 (antibody 19C7), and also demonstrates that the recognition of CTRL1 1505 (BSA-P1 epitopic peptide control) by the antibody 19C7 (P1 1500) is not affected by the presence of troponin I in the sample.

### BIBLIOGRAPHIC REFERENCES

[1] BRULS et al. “Rapid integrated biosensor for multiplexed immunoassays based on actuated magnetic nanoparticles”. The Royal Society of Chemistry, (2009). 3504, Lab Chip, 2009, 9, 3504-3510.
[2] DITTMER et al. “Rapid, high sensitivity, point-of-care test for a cardiac troponin based on optomagnetic biosensor”. Clinica Chimica Acta, (2010). 411 (2010) 868-873.
[3] MORROW et al. Clin. Chem. (Washington D.C.), (2007). 53, 552-574.
[4] JARRIGE et al. “A fast intraoperative PTH point-of-care assay on the Philips handheld magnotech system”. Langenbecks Arch Surg, (2011). 396:337-343.
[5] YAGER et al. Nature, (2006). 442, 412-418.
[6] WILD, D. The Immunoassay Handbook, Elsiever, Amsterdam, 2005.
[7] DITTMER et al. (Journal of Immunological Methods, 338: 40-46).

1. A device making it possible to detect and/or quantify at least one analyte in a liquid sample during a biological analysis employing at least two binding partners P1 and P2 of said analyte, the first binding partner P1 being immobilized within said device and the second binding partner P2 being immobilizable by formation of a sandwich-type complex with the analyte and the first binding partner P1, said device comprising at least two areas in fluid communication, namely:
a) a liquid sample application area, and
b) a reaction area for detecting and/or quantifying said at least one analyte, said device comprising at least a first analogue CTRL1 of said analyte, said first analogue CTRL1 being immobilizable by binding to the immobilized first binding partner P1, and at least a second analogue CTRL2 of the analyte, said second analogue CTRL2 being immobilized within said device so as to bind to the immobilizable second binding partner P2, and thereby immobilize the latter,
said reaction area b) comprising at least the three following regions:
b.1) a first region for detecting and/or quantifying the analyte, by revealing the formation of a sandwich-type complex between the first binding partner P1, the analyte and the second binding partner P2,
b.2) a second biological control region for the first binding partner P1, in which the first binding partner P1 is immobilized, and a bond between the first binding partner P1 and the first analyte analogue CTRL1 is revealed, indicating a positive control for the first binding partner P1, and
b.3) a third biological control region for the second binding partner P2, in which the second analyte analogue CTRL2 is immobilized, and a bond between the second binding partner P2 and the second analyte analogue CTRL2 is revealed, indicating a positive control for the second binding partner P2.

2. The device according to claim 1, said device comprising a liquid sample migration area c), enabling migration of the liquid sample from the liquid sample application area a) toward the reaction area b).

3. The device according to claim 1 or 2, wherein the first and second binding partners P1 and P2 consist in antibodies, and the first and second analyte analogues CRTL1 and CRTL2 comprise at least the epitopes recognized respectively by the first and second antibodies P1 and P2, preferably the first and second analyte analogues CRTL1 and CRTL2 being peptides containing at least the epitopes recognized respectively by the first and second antibodies P1 and P2.

4. The device according to one of claims 1 to 3, wherein the second binding partner P2 and the first analyte analogue CTRL1 are initially deposited into the device, and are suitable for being re-suspended in the presence of the liquid sample.

5. The device according to one of claims 1 to 4, wherein the reaction area b) comprises at least two distinct parts, for example two distinct reaction chambers, with no direct fluid communication between the two parts, the first part comprising the first region b.1) and the second part comprising the second and third regions b.2) and b.3).

6. The device according to claim 5, wherein the first part comprises, besides the immobilized first binding partner P1, the immobilizable second binding partner P2, and the second part comprises, besides the first binding partner P1 and the second analyte analogue CRTL2, which are both immobilized, the first analyte analogue CTRL1 and the second binding partner P2, which are both immobilizable.

7. The device according to one of claims 1 to 4, wherein the reaction area b) comprises at least two distinct parts, for example two distinct reaction chambers, with no direct fluid communication between the two parts, the first part comprising the first and third regions b.1) and b.3), and the second part comprising the second region b.2).

8. The device according to claim 7, wherein the first part comprises, besides the first binding partner P1 and the second analyte analogue CRTL2, which are both immobilized, the immobilizable second binding partner P2, and the second part comprises, besides the immobilized first binding partner P1, the immobilizable first analyte analogue CTRL1, the immobilizable second binding partner P2 preferably being present in excess in said first part.

9. The device according to one of claims 1 to 4, wherein the reaction area b) comprises at least three distinct parts, for example three reaction chambers, with no direct fluid communication between the three distinct parts, the first part comprising region b.1), the second part comprising region b.2) and the third part comprising region b.3).

10. The device according to claim 9, wherein the first part comprises, besides the immobilized first binding partner P1, the immobilizable second binding partner P2, the second part comprises, besides the immobilized second analyte analogue CTRL2, the immobilizable second binding partner P2, and the third part comprises, besides the immobilized first binding partner P1, the immobilizable first analyte analogue CTRL1.

11. The device according to one of claims 5 to 10, wherein the parts are positioned in parallel or in series on the liquid sample pathway, preferably in parallel.

12. The device according to one of claims 1 to 4, wherein the reaction area b) comprises at least one part, for example a reaction chamber, said part comprising:
the three regions b.1), b.2) and b.3),
the second binding partner P2 and the first analyte analogue CTRL1, which are both immobilizable,
the first and second binding partners P1 and P2 being present in excess, and wherein:
the first analyte analogue CTRL1 is associated, directly or indirectly, with a label M1 making it possible to reveal the binding of said first analyte analogue CTRL1 with the first binding partner P1 in regions b.1) and b.2), and/or
the second binding partner P2 being associated, directly or indirectly, with a label M2, different from label M1, making it possible to reveal the formation of the sandwich-type complex between the second binding partner P2, the analyte and the first binding partner P1 in regions b.1) and b.2).

13. The device according to one of claims 2 to 12, wherein the liquid sample migration area c) comprises a matrix.

14. The device according to one of claims 1 to 13, wherein the liquid sample application area a) comprises a filter.

15. A method for detecting and/or quantifying at least one analyte in a liquid sample during a biological analysis employing at least two analyte binding partners P1 and P2, said method comprising the steps consisting in:
(i) placing the liquid sample in contact with a device as defined in any one of claims 1 to 14,
(ii) interpreting the result obtained from said device if the biological control of the binding partner P1 and the biological control of the binding partner P2 are positive, respectively in regions b.2) and b.3),
(iii) otherwise, considering the result obtained as uninterpretable.

16. Use of at least two analyte analogues CTRL1 and CTRL2 for the biological control, respectively, of at least two binding partners P1 and P2, said binding partners P1 and P2 making it possible to detect and/or quantify said analyte by revealing the formation of a sandwich-type complex between the second binding partner P2, the analyte and the first binding partner P1.
